(19) United States
(12) Patent Application Publication
(10) Pub. No.: US 2018/0199551 A1
(43) Pub. Date: Jul. 19, 2018

(54) Process for producing young chicken

(71) Applicant: HatchTech Group B.V., Veenendaal (NL)

(72) Inventor: Tjitze Meter, Veenendaal (NL)

(73) Assignee: HatchTech Group B.V., Veenendaal, OT (NL)

(21) Appl. No.: 15/121,386

(22) PCT Filed: Feb. 25, 2015
(86) PCT No.: PCT/NL2015/050116

§ 371 Date: Aug. 25, 2016

(30) Foreign Application Priority Data
Feb. 25, 2014 (NL) 2012322

(51) Int. Cl. A01K 67/02; A01K 31/18; A01K 39/012; G01N 33/08; A01K 41/02; A01K 41/04; A01K 43/08; A01K 43/10

(52) U.S. Cl. CPC A01K 67/02; A01K 31/18; A01K 39/012; A01K 43/10; A01K 41/023; A01K 41/04; A01K 43/08; G01N 33/085

(57) ABSTRACT

The invention relates to a method for producing chicken at a hatchery, the method comprising;
incubating a batch of eggs in an incubating device during an incubating period of between about 7 to about 20 days, and then,
for each egg of the batch, detecting the presence of a hatchable chicken embryo,
based on the presence of a hatchable chicken embryo, dividing the batch of eggs into at least a hatchable batch portions and a not-hatchable batch portion,
hatching the hatchable batch portion until hatching occurs,
rearing of chicken hatched from said hatchable batch portion,
   wherein the method comprises at the hatchery processing the not-hatchable batch portion into feed and administering said feed at the hatchery to hatched chickens during rearing.

### BACKGROUND

The present invention relates to a method for producing chicken at a hatchery, the method comprising;
incubating a batch of eggs in an incubating device during an incubating period of between about 7 to about 20 days, and then
for each egg of the batch, detecting the presence of a hatchable chicken embryo,
dividing the batch of eggs into at least a hatchable batch portions and a not-hatchable batch portion,
hatching the hatchable batch portion until hatching occurs,
rearing of chicken hatched from said hatchable batch portion.

In this connection WO2007110861, titled APPARATUS AND METHOD FOR FEEDING CHICKS DURING THE HATCHING PROCESS, discloses a method and a compartment within an incubator, capable of allowing chicks to feed during the hatching process without reducing hatchability or liveability. Thanks to immediate post hatch nutrition, the development during the chicks first days is significantly improved, resulting in a significant improvement in their weight as well.

It is known in the art to dispose not hatchable eggs of the hatchery and process these not hatchable eggs into animal feed.

### SUMMARY OF THE INVENTION

The invention aims to provide a method for producing chicken at a hatchery wherein the method has a reduced number of in- and outgoing process flows.

Another object of the invention is to improve a method for producing chicken at a hatchery in that a problem associated with known methods is at least partly solved.

Yet another object of the invention is to provide an alternative method for producing chicken at a hatchery.

According to a first aspect of the invention this is realized with a method for producing chicken at a hatchery, the method comprising;
incubating a batch of eggs in an incubating device during an incubating period of between about 7 to about 20 days, and then,
for each egg of the batch, detecting the presence of a hatchable chicken embryo,
based on the presence of a hatchable chicken embryo, dividing the batch of eggs into at least a hatchable batch portions and a not-hatchable batch portion,
hatching the hatchable batch portion in a hatching device until hatching occurs,
rearing of chicken hatched from said hatchable batch portion,
   wherein the method comprises at the hatchery processing the not-hatchable batch portion into feed and administering said feed at the hatchery to hatched chickens during rearing.

The at the hatchery processing the not-hatchable batch portion into feed and administering said feed at the hatchery to hatched chickens during rearing reduces the number of in- and outgoing process flows because at the hatchery an ingoing flow of feed as well as an outgoing flow of not-hatchable eggs is at least partly omitted.

“At the hatchery” defines a site where eggs are hatched to chicks between 0 to 4 days old. Thereafter, these 0 to 4 days old chicks are transported away from the hatchery to breeding farms where the chicks grow further. It is conceivable that incubating a batch of eggs in an incubating device during an incubating period of between about 7 to about 20 days, takes place somewhere else remote from the hatchery.

Detecting the presence of a hatchable chicken embryo, refers to a step which is known in the art as candling of eggs. The presence of an embryo is detected from the outside of the egg. The detecting step leads to an estimate whether the embryo may be successfully hatched or not. In other words the egg is hatchable or not.

In this respect, rearing refers to initial rearing shortly after hatching.

In an embodiment the method comprises administering only said feed at the hatchery, to hatched chickens during rearing, such that additional nutrients are not required at least for an initial rearing period of about 1 to 4 days. This even more omits an ingoing flow of feed. It will be clear that “only” refers to said feed. There may be other matters administered to the chicken as desired, like medicine.

In an embodiment, the method comprises at the hatchery processing the entire not-hatchable batch portion into feed and administering said feed at the hatchery to hatched chickens during rearing such that no process flow of eggs or feed leaves the hatchery. This even more omits an outgoing flow of not-hatchable eggs.

In an embodiment, the rearing of chicken hatched from said hatchable batch portion comprises rearing the newly hatched chickens in the hatching device, and the administering said feed at the hatchery to hatched chickens comprises administering said feed in the hatching device during hatching. The administering said feed in the hatching device during hatching facilitates early feeding which promotes chick quality. During hatching means that feed is already administered if hatching starts.

In an embodiment, the method comprises rearing the newly hatched chicken in the hatching device during an initial rearing period of between 0 to 4 days.

In an embodiment of the method, the at the hatchery processing the not-hatchable batch portion into feed comprises sterilizing eggs and/or egg components. This even more facilitates the processing of the not-hatchable batch portion into feed.

In an embodiment of the method, the at the hatchery processing the not-hatchable batch portion into feed comprises one or more of;
selecting eggs,
breaking eggs to obtain a mixture of egg shells and egg contents,
separating egg contents into shell, yolk and albumen,
disinfecting egg components,
cooking eggs,
processing eggs into a crumbly substance,
pelletizing eggs.

In an embodiment, the at the hatchery processing the not-hatchable batch portion into feed comprises adding nutrients and/or other animal feed substances for supplementing said feed.

In an embodiment, the administering said feed at the hatchery to hatched chickens comprises administering said feed to chickens hatched from said hatchable batch portion of eggs.

In an embodiment, the administering said feed at the hatchery to hatched chickens comprises administering said feed to chickens hatched from a subsequent or preceding batch of eggs. This allows to take into account the time needed to process the not-hatchable batch portion into feed.

In an embodiment of the method, the detecting the presence of a hatchable chicken embryo comprises one or more of heartbeat detection with respect to an egg, candling of an egg, thermal imaging of an egg, colour measurement of an egg, weighing of an egg. Preferably, then the detecting is followed by, based on the presence of a not-hatchable chicken embryo and colour or weight of an egg, dividing the not-hatchable batch portion into at least a first not-hatchable batch portion and a second not-hatchable batch portion, wherein eggs of the first not-hatchable batch portion are non-rotten and contain no embryo or an embryo smaller than about 20 mm, and eggs of the second not-hatchable batch portion are rotten and/or contain an embryo larger than about 20 mm.

In an embodiment, the method comprises processing the first not-hatchable batch portion into feed.

The invention further relates to a device comprising one or more of the characterising features described in the description and/or shown in the attached drawings.

The invention further relates to a method comprising one or more of the characterising features described in the description and/or shown in the attached drawings.

The various aspects discussed in this patent can be combined in order to provide additional advantages.

### DESCRIPTION OF THE DRAWINGS

The invention will be further elucidated referring to the drawings wherein shown in:

FIG. 1 a flow diagram of a known method for producing chicken at a hatchery; and

FIG. 2 a flow diagram of a methods for producing chicken according to the invention;

### DETAILED DESCRIPTION OF EMBODIMENTS

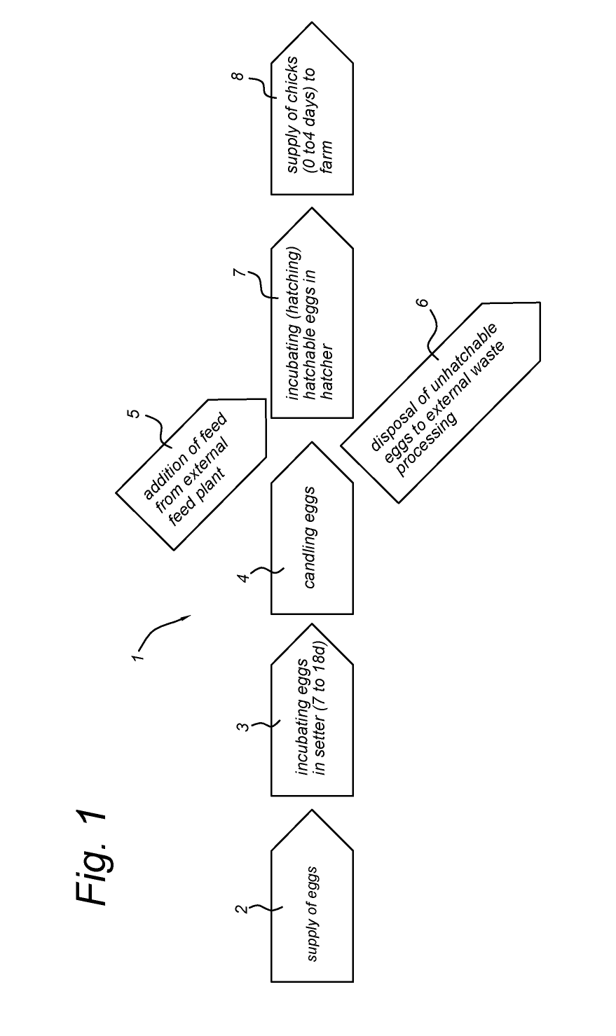

In the FIG. 1 a known method 1 for producing chicken at a hatchery is shown. A main flow 2,3,4,7,8, and secondary flows 5 and 6 can be distinguished. The main flow is the process at the hatchery from egg to newly hatched chicken. One secondary flows is the ingoing flow 5 of feed from an external feed plant away from the hatchery. Another secondary flow is the outgoing flow 6 of unhatchable eggs to an external waste processing away from the hatchery.

The main flow 2,3,4,7,8 will now be described. A batch of eggs is supplied 2 from a site to the hatchery. The batch of eggs is then incubated 3 in an incubating device like a setter during an incubating period of between about 7 to about 20 days.

Subsequently, for each egg of the batch of eggs, the presence of a hatchable chicken embryo is detected 4 by using candling technology which is known in the art of producing chicken. Any suitable technology will suffice, like e.g. heartbeat detection with respect to an egg, and thermal imaging of an egg.

Based on the outcome of the detection, thus on the presence of a hatchable chicken embryo, the batch of eggs is divided into a hatchable batch portions and a not-hatchable batch portion. The hatchable batch portion is then hatched 7 until hatching occurs.

Finally, the method 1 ends when the chicken that have hatched from the hatchable batch portion are transported 8 to a breeding farm away from the hatchery. The chicken are between 0 to 4 days when they leave the hatchery.

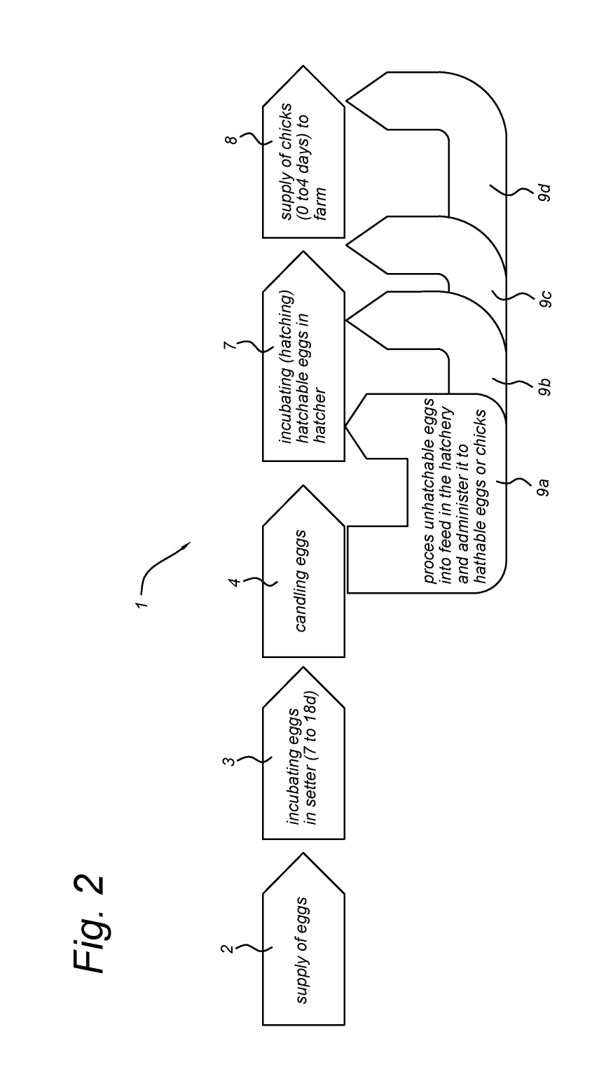

FIG. 2 shows a flow diagram of a method 1 for producing chicken according to the invention. In general only differences compared with the known method of FIG. 1 are described.

As shown according to the invention an ingoing flow of feed as well as an outgoing flow of not-hatchable eggs is omitted.

Instead, the method 1 comprises at the hatchery processing 9a the not-hatchable batch portion into feed. The feed is then administering 9b, 9c, 9d at the hatchery to hatched chickens during rearing. Here, the feed is administered 9b in the hatching device during hatching which promotes taking in of feed by newly hatched chicken as soon as possible.

In this case only feed originating from the not-hatchable batch portion is administered at the hatchery such that a closed system is obtained with respect to feed. This eases the process in terms of traceability and transportation. No or less process flow of eggs or feed leaves the hatchery. In addition, costs are reduced since the not-hatchable batch portion is recycled.

Preferably said feed is administered to chickens hatched from said hatchable batch portion of eggs, it is however conceivable that said feed is administered to chickens hatched form a subsequent batch of eggs. As will be understood, the processing 9a of the not-hatchable batch portion into feed will take its time of passage parallel to the main flow 2, 3, 4, 7, 8.

It will also be obvious after the above description and drawings are included to illustrate some embodiments of the invention, and not to limit the scope of protection. Starting from this disclosure, many more embodiments will be evident to a skilled person which are within the scope of protection and the essence of this invention and which are obvious combinations of prior art techniques and the disclosure of this patent.

1.-13. (canceled)

14. A method for producing chicken at a hatchery, the method comprising;
incubating a batch of eggs in an incubating device during an incubating period of between about 7 to about 20 days, and then,
for each egg of the batch, detecting the presence of a hatchable chicken embryo,
based on the presence of a hatchable chicken embryo, dividing the batch of eggs into at least a hatchable batch portion and a not-hatchable batch portion,
hatching the hatchable batch portion in a hatching device until hatching occurs,
rearing of chicken hatched from said hatchable batch portion,
processing the not-hatchable batch portion into feed, and
administering said feed at the hatchery to hatched chickens.

15. The method according to claim 1, and further comprising administering only said feed at the hatchery to the hatched chickens during rearing.

16. The method according to claim 15, wherein the step of administering only said feed at the hatchery to the hatched chickens comprises administering only said feed at the hatchery to the hatched chickens for an initial rearing period of 1 to 4 days.

17. The method according to claim 1, wherein the steps of processing the entire not-hatchable batch portion into feed and administering said feed at the hatchery to hatched chickens during rearing are performed at the hatchery such that no process flow of eggs or feed leaves the hatchery.

18. The method according to claim 1, wherein the step of rearing of chicken hatched from said hatchable batch portion comprises rearing the newly hatched chickens in the hatching device, and the step of administering said feed at the hatchery to hatched chickens comprises administering said feed in the hatching device during hatching.

19. The method according to claim 18, and further comprising rearing the newly hatched chicken in the hatching device during an initial rearing period of 0 to 4 days.

20. The method according to claim 1, wherein the step of processing the non-hatchable batch portion into feed comprises sterilizing eggs and/or egg components.

21. The method according to claim 20, wherein the step of processing the not-hatchable batch portion into feed comprises one or more of:
selecting eggs,
breaking eggs to obtain a mixture of egg shells and egg contents,
separating egg contents into shell, yolk and albumen,
disinfecting egg components,
cooking eggs,
processing eggs into a crumbly substance, and
pelletizing eggs.

22. The method according to claim 1, and further comprising adding nutrients and/or other animal feed substances for supplementing said feed.

23. The method according to claim 1, wherein the administering said feed at the hatchery to hatched chickens comprises administering said feed to chickens hatched from said hatchable batch portion of eggs.

24. The method according to claim 1, wherein administering said feed at the hatchery to hatched chickens comprises administering said feed to chickens hatched from a subsequent or preceding batch of eggs.

25. The method according to claim 1, wherein the step of detecting the presence of a hatchable chicken embryo comprises one or more of: heartbeat detection with respect to an egg, candling of an egg, thermal imaging of an egg, colour measurement of an egg, and weighing of an egg.

26. The method according to claim 25, and further comprising dividing the not-hatchable batch portion into at least a first not-hatchable batch portion and a second not-hatchable batch portion based on the presence of a not-hatchable chicken embryo and colour or weight of an egg.

27. The method according to claim 26, wherein eggs of the first not-hatchable batch portion are non-rotten and contain no embryo or an embryo smaller than about 20 mm, and eggs of the second not-hatchable batch portion are rotten and/or contain an embryo larger than about 20 mm.

28. The method according to claim 26, and further comprising processing the first not-hatchable batch portion into feed.

29. A method for producing feed, the method comprising:
incubating a batch of eggs;
detecting whether each egg in the batch of eggs comprises a hatchable chicken embryo;
removing the eggs which do not comprise a hatchable chicken embryo; and
processing the eggs which do not comprise a hatchable chicken embryo into feed.

30. The method of claim 29, wherein the step of processing the eggs which do not comprise a hatchable chicken embryo into feed comprises one or more of:
sterilizing the eggs and/or egg components;
selecting eggs which are not rotten and/or which contain no embryo or an embryo smaller than 20 mm;
breaking the eggs to obtain a mixture of egg shells and egg contents;
separating the egg contents into shell, yolk and albumen;
disinfecting the egg components;
cooking the eggs;
processing the eggs into a crumbly substance, and
pelletizing the eggs.

31. The method according to claim 29, and further comprising adding nutrients and/or other animal feed substances for supplementing said feed.

32. The method according to claim 29, wherein the step of detecting whether each egg in the batch of eggs comprises a hatchable chicken embryo comprises one or more of: heartbeat detection with respect to an egg, candling of an egg, thermal imaging of an egg, colour measurement of an egg, and weighing of an egg.

33. The method of claim 29, wherein the step of incubating a batch of eggs is performed at a different location than one or more of the other steps.
